(19) United States
(12) Patent Application Publication
(10) Pub. No.: US 2024/0345057 A1
(43) Pub. Date: Oct. 17, 2024

(54) FILM SENSOR AND HYDROGEN DETECTION SYSTEM

(71) Applicant: TOHOKU UNIVERSITY, Sendai-shi, Miyagi (JP)

(72) Inventors: Hiroshi KAKINUMA, Sendai-shi (JP); Eiji AKIYAMA, Sendai-shi (JP); Saya AJITO, Sendai-shi (JP); Motomichi KOYAMA, Sendai-shi (JP); Tomohiko HOJO, Sendai-shi (JP)

(21) Appl. No.: 18/293,613

(22) PCT Filed: Aug. 2, 2021
(86) PCT No.: PCT/JP2021/028591

§ 371 Date: Jan. 30, 2024

(51) Int. Cl. G01N 33/2025; G01N 21/64; G01N 21/78

(52) U.S. Cl. CPC G01N 33/2025; G01N 21/643; G01N 21/78; G01N 2021/6439

(57) ABSTRACT

A film sensor for detecting hydrogen in a metal material by being laminated on a surface of the metal material in a gas or in vacuum is formed of an organic polymer whose optical characteristic changes depending on a hydrogen concentration, and contains, if necessary, a dye whose optical characteristic changes depending on the hydrogen concentration. This film sensor can easily detect a concentration or a distribution of hydrogen present in the metal material, and additionally enables observation of change over time of the concentration or the distribution of hydrogen.

### TECHNICAL FIELD

The present invention relates to a film sensor and a hydrogen detection system for detecting hydrogen in a metal material.

### BACKGROUND ART

There is a concern that various metal materials such as high-strength steel, Al, Ti, and Zr alloys may cause hydrogen to intrude in an environment where being used, such as atmospheric corrosion, high-temperature water, sour gas, and high-pressure hydrogen gas, and may cause hydrogen embrittlement.

In particular, it is known that, in steel materials, sensitivity of the hydrogen embrittlement rises as strength increases. Hydrogen that has intruded the material is localized in a stress concentration portion or the like to cause a breakdown phenomenon.

For this reason, it is indispensable to understand the concentration and the presence location of hydrogen present in the material in order to clarify a hydrogen embrittlement mechanism and to prevent the embrittlement. In addition, there are two types of hydrogen intrusion processes, intrusion from a gas phase represented by a high-pressure hydrogen gas environment and intrusion from a liquid phase accompanying a hydrogen generation reaction generated in an atmospheric corrosion process, and a rate of the hydrogen intrusion from the liquid phase differs greatly depending on an atmospheric environment. Therefore, in order to prevent the destruction caused by hydrogen, it is necessary to provide a hydrogen detector capable of continuously measuring the local accumulation of hydrogen in a material and capturing it as a still image or a video.

In addition, in a field where a metal material is used, a means for easily grasping an embrittlement state is required.

The silver decoration method (see, for example, Non-PTL 1) or the hydrogen microprinting method (for example, see PTL 1) is known as a method for visualizing the presence location of hydrogen in a material. In the silver decoration method, a material containing hydrogen is immersed in a potassium silver(I) cyanide aqueous solution, so that silver particles are reduced and precipitated in a location where the hydrogen atoms exposed on the surface of a material are ionized and released.

Further, in the hydrogen microprinting method, by coating the surface of a material containing hydrogen with an emulsion containing silver(I) bromide and gelatin as main components, and by reducing silver ions, adsorbed hydrogen on the surface of the material is ionized, and locations where adsorbed hydrogen exists can be visualized from a distribution of silver particles that are reduced and precipitated.

In addition, PTL 2 discloses that a metal oxide layer is provided on the surface of a metal material via a hydrogen buffer film, and a concentration of hydrogen present in the metal material or hydrogen intruding from outside and a change with time of a presence location are measured from visible and ultraviolet reflection spectrum of the metal oxide layer which changes according to a hydrogen concentration of the material.

### CITATION LIST

### Patent Literature

PTL 1: JP2006-258595A

PTL 2: JP2018-13424A

### Non Patent Literature

Non-PTL 1: T. Schober and C. Dieker, “Observation of Local Hydrogen on Nickel Surfaces”, Metall. Trans. A, 1983, 14A, p. 2440

### SUMMARY OF INVENTION

### Technical Problem

In both of the silver decoration method of Non-PTL 1 and the hydrogen microprinting method of PTL 1, since the presence location of hydrogen is visualized from the distribution of precipitated silver particles, a change with time of the hydrogen concentration cannot be measured.

Further, there is a problem that the silver decoration method needs to use a cyanide, which is dangerous.

The metal oxide layer of PTL 2 must be formed by a sputtering method or a vapor deposition method, which uses a large-sized apparatus. Further, the hydrogen buffer film needs to be formed by the sputtering method, the vapor deposition method, or the like. Therefore, it is difficult to apply the method of PTL 2 to a metal material already used in a field. In addition, in the case of sputtering, an area that can be formed into a film is small, making it difficult to form a film with a sufficient area necessary for hydrogen detection in metal materials in the field.

In view of the above circumstances, the object of the invention is to provide a film sensor and a hydrogen detection system capable of easily detecting hydrogen present in a metal material.

### Solution to Problem

In order to achieve the above object, the invention employs the following configurations.
[1] A film sensor for detecting hydrogen in a metal material by being laminated on a surface of the metal material in a gas or in vacuum, in which
the film sensor is formed of an organic polymer whose optical characteristic changes depending on a hydrogen concentration.
[2] The film sensor according to [1], in which
the organic polymer whose optical characteristic changes depending on the hydrogen concentration is a conjugated polymer.
[3] The film sensor according to [2], in which
the organic polymer whose optical characteristic changes depending on the hydrogen concentration is an aromatic conjugated polymer.
[4] The film sensor according to any one of [1] to [3], in which
the organic polymer whose optical characteristic changes depending on the hydrogen concentration has at least one of an amine structure or an imine structure.
[5] The film sensor according to any one of [1] to [4], in which
the film sensor contains a dye whose optical. characteristic changes depending on the hydrogen concentration.
[6] A film sensor for detecting hydrogen in a metal material by being laminated on a surface of the metal material in a gas or in vacuum, in which
the film sensor contains a dye which is formed of an organic polymer and whose optical characteristic changes depending on a hydrogen concentration.
[7] A hydrogen detection system including:
the film sensor according to any one of claims [1] to [6]; and
an optical detector configured to detect an optical characteristic of the film sensor.

The invention includes the following aspects.
[1] A film sensor for detecting hydrogen in a metal material by being laminated on a surface of the metal material in a gas or in vacuum, in which
the film sensor is formed of an organic polymer whose optical characteristic changes depending on a hydrogen concentration, and
a film thickness of the film sensor is 0.1 μm or more, preferably 0.5 μm or more.
[2] The film sensor according to [1], in which
the film thickness of the film sensor is 1 mm or less, preferably 500 μm or less, more preferably 100 μm or less, still more preferably 5 μm or less, particularly preferably 1 μm or less.
[3] The film sensor according to [1] or [2], in which
the organic polymer whose optical characteristic changes depending on the hydrogen concentration is a conjugated polymer, preferably an aromatic conjugated polymer.
[4] The film sensor according to any one of [1] to [3], in which
the organic polymer whose optical characteristic changes depending on the hydrogen concentration is a compound having at least one of an amine structure or an imine structure, preferably at least one selected from the group consisting of: aromatic conjugated polymers such as polyaniline, poly(σ-methoxyaniline), poly((σ-5-toxianiline), and polypyrrole; poly(ether tert-amine); poly(N-isopropyl acrylic amide-co-acrylic acid);, and poly(amine-ether).
[5] The film sensor according to any one of [1] to [4], in which
the film sensor contains a dye whose optical characteristic changes depending on the hydrogen concentration.
[6] The film sensor according to [6], in which
the dye whose optical characteristic changes depending on the hydrogen concentration is at least one selected from the group consisting of methyl red, phenolphthalein, p-nitrophenylhydrazone, alizarin yellow, and sodium fluorescein.
[7] The film sensor according to [5] or [6], in which
an amount of 0.01 g to 5 g, preferably 0.01 g to 0.05 g of the dye whose optical characteristic changes depending on the hydrogen concentration is contained with respect to 50 g of the organic polymer whose optical characteristic changes depending on the hydrogen concentration.
[8] A film sensor for detecting hydrogen in a metal material by being laminated on a surface of the metal material in a gas or in vacuum, in which
the film sensor contains a dye which is formed of an organic polymer and whose optical characteristic changes depending on a hydrogen concentration.
[9] The film sensor according to [8], in which the dye whose optical characteristic changes depending on the hydrogen concentration is at least one selected from the group consisting of methyl red, phenolphthalein, p-nitrophenylhydrazone, alizarin yellow, and sodium fluorescein.
[9] The film sensor according to [8] or [9], in which an amount of 0.01 g to 5 g, preferably 0.01 g to 0.05 g of the dye whose optical characteristic changes depending on the hydrogen concentration is contained with respect to 50 g of the organic polymer whose optical characteristic changes depending on the hydrogen concentration.
[9] The film sensor according to any one of [1] to [10], in which
the film sensor is laminated on the surface of the metal material in a gas or in vacuum.
[11] The film sensor according to [11], in which the film sensor is laminated so as to be in direct contact with the surface of the metal material.
[12] A hydrogen detection system including:
the film sensor according to any one of claims [1] to [10]; and
an optical detector configured to detect an optical characteristic of the film sensor.
[14] The hydrogen detection system according to [13], in which
[15]0 the film sensor is laminated on the surface of the metal material in a gas or in vacuum.

The hydrogen detection system according to [14], in which
the film sensor is laminated so as to be in direct contact with the surface of the metal material.

[16] A method for detecting hydrogen in the metal material including:
visually observing the film sensor according to or [12].
[17]A method for detecting hydrogen in the metal material including:
observing the film sensor with the optical detector in the hydrogen detection system according to or [15].

### Advantageous Effects of Invention

According to the film sensor and the hydrogen detection system of the invention, hydrogen present in a metal material can be easily detected.

### BRIEF DESCRIPTION OF DRAWINGS

FIG. 1 is a schematic diagram showing a method of forming a film sensor on a pipe.

FIG. 2 (a) of FIG. 2 is a schematic front view of a hydrogen detection system used in Experimental Example 1 and the like of the invention, and (b) of FIG. 2 is a schematic view of a cell opening side of an acrylic cell as viewed from an inside of an acrylic cell 10.

FIG. 3 shows images captured by a camera when predetermined times elapse after starting constant current polarization in an apparatus of FIG. 2.

FIG. 4 is a graph showing the amount of change in an RGBY value after starting the constant current polarization in the apparatus of FIG. 2.

FIG. 5 is a graph showing the amount of change in the R value after the constant current polarization ends in the apparatus of FIG. 2.

FIG. 6 is a graph showing stability of a film sensor 2 in an atmosphere.

FIG. 7 (a) of FIG. 7 is a schematic front view of a hydrogen detection system used in Experimental Example 5 of the invention, and (b) of FIG. 7 is a schematic view of a cell opening side of an acrylic cell as viewed from the inside of an acrylic cell 10.

FIG. 8 shows images captured by a camera when predetermined times elapse after starting constant current polarization in an apparatus of FIG. 7.

### DESCRIPTION OF EMBODIMENTS

In the present description and claims, a description that a film sensor “is laminated on a surface of a metal material in a gas or in vacuum” means that an interface between the metal material and the film sensor, or an interface between a layer that may exist between the metal material and the film sensor and the film sensor is present in a gas or in vacuum and is not present in a liquid or a solid.

In the present description and claims, a description of “change in optical characteristics” includes not only changes such as discoloration, color development, and decoloring, but also light emission such as fluorescence emission or light absorption. In addition, the change in optical characteristics is not limited to a change in a visible region, and may be for example, light emission or light absorption in a near-infrared region.

In the present description and claims, “to” indicating a numerical range means that numerical values described before and after “to” are included as a lower limit value and an upper limit value.

### First Embodiment

A film sensor according to a first embodiment is formed of an organic polymer whose optical characteristics change depending on a hydrogen concentration (hereinafter, may be referred to as a “hydrogen functional polymer”). The film sensor according to the first embodiment may contain a dye whose optical characteristics change depending on the hydrogen concentration (hereinafter, may be referred to as a “hydrogen functional dye”).

The film sensor according to the first embodiment can detect hydrogen in a metal material by being laminated on a surface of the metal material in a gas or in vacuum.

### Hydrogen Functional Polymer

A hydrogen functional polymer used for the film sensor may be an organic polymer whose optical characteristics change depending on the hydrogen concentration.

The hydrogen functional polymer is preferably a conjugated polymer and more preferably an aromatic conjugated polymer, since it is easy to obtain an optical characteristic change thereof depending on the hydrogen concentration.

The hydrogen functional polymer preferably has at least one of an amine structure or an imine structure since a larger optical characteristic change can be obtained depending on the hydrogen concentration.

Examples of compounds having at least one of an amine structure or an imine structure include: aromatic conjugated polymers such as polyaniline, poly (σ-methoxyaniline), poly (σ-5-toxianiline), and polypyrrole; poly (ether tert-amine); poly (N-isopropyl acrylic amide-co-acrylic acid); and poly (amine-ether).

Examples of a hydrogen functional polymer having neither an amine structure nor an imine structure include poly (methylacrylic acid), 11-methacrylamide undecanoic acid, 2-acrylamide-2-methylpropane sulfonic acid, and polythiophene.

The hydrogen functional polymer itself preferably has film formability. Accordingly, a film sensor can be formed without using other polymers.

Further, the hydrogen functional polymer having the film formability is not limited to a hydrogen functional polymer formed in a film shape as it is when polymerized, but may also be a hydrogen functional polymer formed in a powder shape. The hydrogen functional polymer formed in the powder shape can be formed into a film by filtration or drying.

In order to have the film formability, the number average molecular weight is required to be at least 4000 or more. In addition, the number average molecular weight is required to exceed a critical molecular weight. The critical molecular weight differs depending on a type of the polymer.

Two or more kinds of hydrogen functional polymers may be used in combination. In addition, organic polymers which are not hydrogen functional polymers may be used in combination as long as the effect of the invention is not impaired.

### Hydrogen Functional Dye

By containing a hydrogen functional dye in a film formed of a hydrogen functional polymer, it is possible to increase the optical characteristic change and improve sensitivity.

A dye usually used as a pH indicator can be used as a hydrogen functional dye.

Examples of the pH indicator include a one-color indicator that develops a color or decolors a color depending on a hydrogen ion concentration, a two-color indicator that discolors depending on the hydrogen ion concentration, and a light emission indicator that emits light depending on the hydrogen ion concentration.

Examples of the one-color indicator include phenolphthalein, picric acid, 2,6-dinitrophenol, para-nitrophenol, tymolphthalein, 2,4,6-trinitrotoluene, 1,3,5-trinitrobenzene, 2,4-dinitrophenol, 2,5-dinitrophenol, and o-nitrophenol.

Examples of the two-color indicator include methyl violet, crystal violet, ethyl violet, methyl green, cresol red, methyl red, paramethyl red, thymol blue, metacresol purple, methyl yellow, bromophenol blue, congo red, methyl orange, ethyl orange, bromocresol green, bromocresol blue, litmus (azolitomine), propyl red, methyl purple, chlorophenol red, bromocresol purple, alizarin, bromothymol blue, brilliant yellow, neutral red, phenol red, para-α-naphthalin, metacresol purple, alizarin yellow R, alizarin yellow G, alizarin red, quinotricarbocyanine-based dyes, metanil yellow, 4-phenylazo diphenylamine, naphthyl red hydrochloride, lacmoid, rosolic acid, α-naphtholphthalein, p-xylenol blue, o-cresolphthalein, o-cresol red, p-naphtholbenzein, tetryl, tropeolin, indigo carmine, malachite green, dimethyl yellow, γ-dinitrophenol, nile blue, nitrothymol yellow, tetrabromophenol blue, p-nitrophenylhydrazone, N,N-dimethylaniline, and anthocyanin.

Examples of the light emission indicator include fluorescent indicators such as sodium fluorescein, 6-carboxyfluorescein, 2′, 7′-bis (2-carboxyethyl)-5-(and-6)-carboxyfluorescein, 8-hydroxypyren-1,3,6-trisulfonic acid, semi-naphthophorofluorescein seminaphthofluorescein, and boron dipyrromethene.

Two or more kinds of hydrogen functional dyes may be used in combination.

When the film sensor contains the hydrogen functional dye, the content of the hydrogen functional dye contained in the film sensor is preferably 0.01 g to 5 g, and more preferably 0.01 g to 0.05 g with respect to 50 g of a hydrogen-sensitive polymer.

### [Film Thickness]

The film sensor can form a film without any practical problem if a film thickness is 1 mm or less. The film thickness of the film sensor is preferably 500 μm or less, more preferably 100 μm or less, still more preferably 5 m or less, and particularly preferably 1 m or less.

As long as the film thickness of the film sensor is equal to or less than the preferable upper limit value, manufacturing becomes easy, and it is also advantageous in terms of cost. In addition, since the movement of hydrogen in the thickness direction of the film is limited, sensitivity tends to be good.

Further, the film sensor can be formed to have light transmissivity by setting the film thickness to 5 μm or less, preferably 1 μm or less.

When the film sensor has light transmissivity, a surface state of the metal material can be observed from above the film sensor. Therefore, it is easy to grasp a correspondence relationship between a change in the optical characteristics of the film sensor and a change in state of the metal material.

The film thickness of the film sensor is preferably 0.1 m or more, and more preferably 0.5 m or more. As long as the film thickness of the film sensor is equal to or more than the preferable lower limit value, a change in optical characteristics due to hydrogen is sufficiently obtained.

When the light transmissivity is not considered, the film thickness of the film sensor is preferably 0.1 μm to 1 mm, more preferably 0.1 μm to 500 μm, still more preferably 0.5 m to 500 m, and particularly preferably 0.5 μm to 100 μm.

When the light transmissivity is considered, the film thickness of the film sensor is preferably 0.1 m to 5 m, more preferably 0.1 μm to 1 m or 0.5 m to 5 m, and particularly preferably 0.5 m to 1 μm.

### Aspect of Film Sensor

Examples of the metal materials in which hydrogen is detected by the film sensor include pure iron, stainless steel, carbon steel, aluminum alloy, and magnesium alloy,

Examples of a location where the metal material is used include pipes for a liquid or a gas, containers such as a high-pressure hydrogen gas cylinder and a natural gas cylinder, and transport machines such as automobiles.

The film sensor can be used as a sensor for detecting hydrogen in a metal material by being laminated on a surface of the metal material in a gas or in vacuum.

The film sensor according to the invention can be laminated so as to be in direct contact with one surface of the metal material. In this case, an interface between the metal material and the film sensor is present in a gas or in vacuum, and is not present in a liquid or a solid. Further, another surface of the metal material on which the film sensor is not laminated (for example, the inner surface of a pipe) may be in contact with a liquid or the like.

The film sensor according to the invention may be laminated on one surface of the metal material via a buffer layer. Examples of the buffer layer include a nickel plating layer, a palladium plating layer, a copper plating layer, and a zinc plating layer. When the buffer layer is provided, an end surface of the buffer layer is present in a gas or in vacuum, and is not present in a liquid or a solid.

When the buffer layer is interposed, there is a tendency that the change in optical characteristics according to the hydrogen concentration becomes large, that is, a tendency that the sensitivity is improved. It is considered that this is because a reaction solution for forming the film sensor does not directly contact the metal material at the time of forming the film sensor, which can prevent the surface of the metal material from becoming rough.

The thickness of the buffer layer is preferably 0.001 μm to 1 μm, and more preferably 0.001 μm to 0.05 m. When the thickness of the buffer layer is equal to or more than the lower limit value of the preferable range, an effect of improving the sensitivity is more easily obtained. When the thickness of the buffer layer is equal to or less than the upper limit value of the preferable range, movement of hydrogen from the metal material to the film sensor is less likely to be inhibited.

Since the optical characteristics of the film sensor are changed due to the hydrogen transferred from the metal material to the film sensor, the hydrogen transferred to the film sensor and thus the hydrogen in the metal material can be detected by observing a surface opposite from the surface on which the film sensor is laminated on the metal material.

Furthermore, it is also possible to evaluate a level of the hydrogen concentration in the metal material and a change of the concentration over time according to the sensitivity of the film sensor.

In addition, by observing a difference in the change of the optical characteristics in the surface on which the film sensor is laminated, it is possible to evaluate a hydrogen distribution in the surface of the metal material on which the film sensor is laminated (the level of the hydrogen concentration depending on a location in the surface).

The change in optical characteristics of the film sensor can be confirmed by visual observation as long as the change is in a visible region. It is also preferable to use a hydrogen detection system in which an optical detector such as a camera is combined with the film sensor. When the optical detector is combined, it is easy to objectively detect the change in optical characteristics, and it is easy to keep a record.

When the change in optical characteristics of the film sensor is not the change in the visible region, the optical detector is necessary. For example, when near-infrared light emission is used, an optical detector having sensitivity in a near-infrared region is necessary.

In the case of a system in which analysis tools for analyzing captured images are combined, the change in optical characteristics can be grasped more objectively.

The change in the optical characteristics of the film sensor can be usually observed under general light such as solar light or an indoor fluorescent lamp, except when excitation light such as fluorescence emission is necessary.

In addition to the film sensor and the optical detector, the hydrogen detection system may further include a specific light source. If the specific light source is used, the change in optical characteristics can be easily detected with good reproducibility.

As the light source, a halogen lamp, a xenon lamp, an LED light, or the like can be used.

The wavelength of the excitation light for obtaining the fluorescence emission differs depending on the type of a pH functional dye, in some cases, an ultraviolet light source is necessary, and in other cases, the general light is sufficient.

### Method for Manufacturing Film Sensor

The film sensor can be obtained by an electrolytic polymerization method or a chemical polymerization method. The electrolytic polymerization method is suitable for stably forming a film sensor having a large area. Since a thin film is easily formed by the chemical polymerization method, the movement of hydrogen in the thickness direction can be prevented, and a high degree of resolution is easily obtained. Therefore, the film sensor obtained by chemical polymerization is suitable for evaluating a narrow area with high sensitivity.

In order to form a film sensor by the electrolytic polymerization method, a reaction solution containing a monomer for obtaining a hydrogen functional polymer or a reaction solution containing a hydrogen functional dye in addition to this monomer may be brought into contact with one surface of a metal material, and may be applied a voltage.

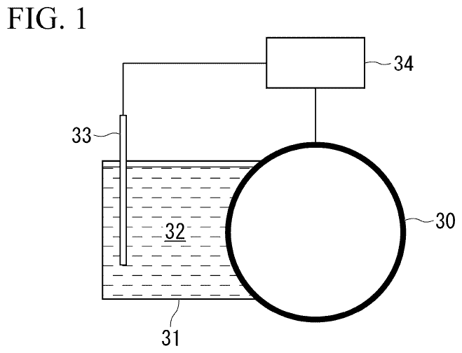

For example, when the film sensor is formed on the surface of a pipe, as shown in FIG. 1, an opening surface of an acrylic cell 31 is pressed against a pipe 30 in a state where liquid tightness is maintained, and a reaction solution 32 is filled into the acrylic cell 31. Then, a voltage from a power source 34 may be applied between the pipe 30 and a counter electrode 33 inserted into the acrylic cell 31 to electrolytically polymerize a monomer in the reaction solution 32.

In order to form a film sensor by the chemical polymerization method, an oxidizing agent is added to a liquid containing a monomer for obtaining a hydrogen functional polymer, or a liquid containing a hydrogen functional dye in addition to the monomer to perform oxidation polymerization, so that a film may be formed by using a dispersion containing a hydrogen functional polymer, or a dispersion containing a hydrogen functional dye in addition to the hydrogen functional polymer. In order to promote the polymerization, the reaction solution may be stirred after adding the oxidizing agent.

Examples of a method for forming a film on one surface of a metal material using a dispersion include a method of filtering a dispersion and drying the dispersion on a metal material, and a method of spraying a dispersion on one surface of a metal material and drying the dispersion on the metal material.

The polymer chemical polymerization reaction is classified into a homogeneous reaction and a heterogeneous reaction, and various reagents are selected according to a purpose.

According to the chemical polymerization method using an oxidizing agent having strong oxidizing power, a hydrogen functional polymer is obtained by the homogeneous reaction. In this case, the hydrogen functional polymer is generated in the form of fine particles in an aqueous solution, and the reaction proceeds more rapidly.

Examples of oxidizing agents having strong oxidizing power include potassium dichromate, potassium iodate, iron(III) chloride, and potassium permanganate.

On the other hand, according to the chemical polymerization method using an oxidizing agent having weak oxidizing power, a hydrogen functional polymer is obtained by the heterogeneous reaction. In this case, since the polymerization proceeds more slowly, a film having high mechanical strength is easily and homogeneously prepared on a surface of a metal material.

Examples of oxidizing agents having weak oxidizing power include ammonium salts such as ammonium peroxodisulfate and ammonium metavanadate, and potassium peroxodisulfate.

In particular, when an ammonium salt is used as the oxidizing agent, it is easy to obtain a homogeneous and high-strength film sensor with high accuracy.

When the dispersion contains an acidic solvent, all or part of the solvent is also preferably replaced with an organic solvent or a neutral aqueous solution. Accordingly, a metal material can be prevented from being corroded by an acid. In addition, since the roughening of a metal surface due to the influence of an acid can be avoided, the sensitivity of the film sensor can be easily improved.

### Second Embodiment

A film sensor according to a second embodiment contains a dye which is formed of organic polymers and whose optical characteristics change depending on the hydrogen concentration (hydrogen functional dye).

The film sensor according to the second embodiment can detect hydrogen in a metal material by being laminated on a surface of the metal material in a gas or in vacuum similarly to the film sensor according to the first embodiment.

The organic polymer in the present embodiment may or may not be a hydrogen functional polymer. An aspect in which a hydrogen functional polymer and an organic polymer which is not a hydrogen functional polymer are used in combination is also included in the embodiment.

In the embodiment, an aspect in which all or part of the organic polymer are hydrogen functional polymers is the same as the aspect containing the hydrogen functional dye according to the first embodiment, and thus, hereinafter, a case where the organic polymer is not a hydrogen functional polymer will be described.

Examples of the organic polymer which is not a hydrogen functional polymer include agar, polystyrene, polyvinyl chloride, butyl rubber, chlorosulfonated polyethylene, nylon, and chloroprene rubber.

The organic polymer which is not a hydrogen functional polymer is required to have a number average molecular weight of at least 4000 or more and a number average molecular weight exceeding a critical molecular weight to make the organic polymer have the film formability. For example, the critical molecular weight of polyethylene oxide is 3200.

Two or more kinds of organic polymers which are not hydrogen functional polymers may be used in combination.

The hydrogen functional dye similar to those described in the first embodiment can be used. The content of the hydrogen functional dye contained in the film sensor is preferably 0.01 g to 5 g, and more preferably 0.01 g to 0.05 g with respect to 50 g of an organic polymer which is not a hydrogen sensitive polymer.

The film sensor according to the present embodiment also preferably has light transmissivity. A preferable film thickness of the film sensor according to the embodiment is the same as that of the first embodiment.

In addition, it is also the same as in the first embodiment that the film sensor can be used as a sensor for detecting hydrogen in a metal material by being laminated on a surface of the metal material in a gas or in vacuum, and that the buffer layer may be provided as necessary.

The film sensor according to the embodiment can also be combined with an optical detector, in addition a light source, and the like to form a hydrogen detection system.

The film sensor according to the embodiment can also be obtained by the electrolytic polymerization method or the chemical polymerization method. In the electrolytic polymerization method, a reaction solution containing a monomer for obtaining an organic polymer and a hydrogen functional dye is used. In the chemical polymerization method, an oxidizing agent is added to a liquid containing a monomer for obtaining a hydrogen functional polymer and a hydrogen functional dye to perform oxidation polymerization, so that a film is formed using filtration or spraying.

### EXAMPLES

Hereinafter, the invention will be specifically described with reference to Examples, but the invention is not limited to the description of these Examples.

### Experimental Example 1

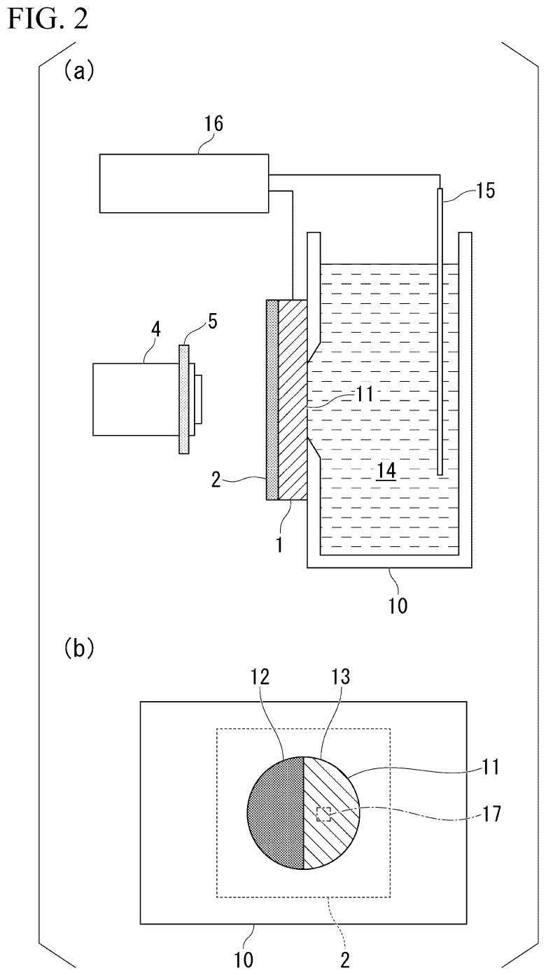

It was confirmed that a hydrogen intrusion state when a metal material was exposed to an electrolytic solution in a state where a voltage was applied could be observed by a hydrogen detection system of FIG. 2.

The hydrogen detection system of FIG. 2 includes a film sensor 2 laminated on one surface of a metal material 1, a camera 4 for imaging the film sensor 2 from a side opposite from the metal material 1 (front side), and a ring light 5 attached to the camera 4, and the entire hydrogen detection system of FIG. 2 is placed in a dark room under an atmospheric environment.

In order to expose the metal material 1 to the electrolytic solution in the state where a voltage was applied, an acrylic cell 10 having a cell opening 11 on a side surface, a potentiostat 16, and a counter electrode 15 connected to the potentiostat 16 were prepared. The metal material 1 was connected to a working electrode side of the potentiostat 16.

Then, a surface of the metal material 1 opposite from a surface on which the film sensor 2 was laminated (back surface) was pressed to a position where the cell opening 11 was to be closed.

As shown in (b) of FIG. 2, half of a portion of the metal material 1 facing the cell opening 11 is a masking portion 12, and the remaining half is an exposed portion 13. The area of the exposed portion 13 was 1 cm². Liquid tightness is maintained between the back surface of the metal material 1 and the acrylic cell 10 by a sealing member (not shown) surrounding the cell opening 11.

In this state, as shown in (a) of FIG. 2, the acrylic cell 10 was filled with an electrolytic solution 14, and a color tone of the film sensor 2 when constant current polarization was performed by the potentiostat 16 was imaged by the camera 4 by lighting the ring light 5 in the dark room. A single-lens reflex camera was used as the camera 4. A halogen lamp was used as a light source of the ring light 5.

As the metal material 1, pure iron having a width of 30 mm, a length of 30 mm, and a thickness of 2 mm and having a purity of 99.5% by mass was thermally treated at 900°° C. in advance, and the front surface and the back surface were polished to mirror surfaces (it is considered that hydrogen is hardly contained) so as to be used.

After the film sensor 2 was formed on a portion to be the masking portion 12 on the back surface of the metal material 1, a masking treatment was performed to avoid contact with the electrolytic solution 14 by coating with a resin (Araldite, manufactured by Huntman Japan Co., Ltd.).

The film sensor 2 was formed on the metal material 1 as follows.

That is, 26.75 mL of sulfuric acid (Fuji Film Wako Pure Chemical Corporation, JIS Special Grade) and 44.6 mL of aniline (Fuji Film Wako Pure Chemical Corporation, JIS Special Grade) were mixed with 800 mL of distilled water, the mixture was stirred until the mixture was completely dissolved, and a mixed solution was prepared by adding distilled water so that a total amount was 1 L. Here, M represents a molar concentration by volume (mol/L).

The metal material 1 in which the back surface was covered with an acid resistant tape (Electroplating Tape 470, manufactured by 3M Japan Ltd.) and the entire front surface is exposed, was immersed in the mixed solution, a voltage of 1 V was applied using a platinum wire as the counter electrode, and held for 200 seconds. A solid polyaniline layer having a thickness of about 5 μm formed on the front surface of the metal was material 1 sufficiently washed with highly pure water and naturally dried at room temperature for 1 hour in an air atmosphere to obtain the film sensor 2.

The diameter of the cell opening 11 of the acrylic cell 10 was 16 mm, and a 3 mass % NaCl aqueous solution was used as the electrolytic solution 14.

As the potentiostat 16, VERTEX 100 mA manufactured by Ivium Technologies B.V. was used. The platinum wire was used as the counter electrode to perform constant current polarization of −1 mA.

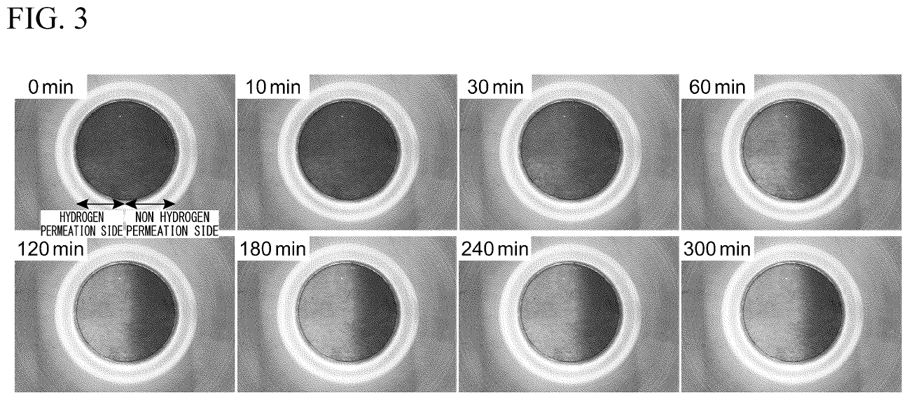

FIG. 3 shows images captured by the camera 4 at the start of the constant current polarization (0 minute), 10 minutes after the start, 30 minutes after the start, 60 minutes after the start, 120 minutes after the start, 180 minutes after the start, 240 minutes after the start, and 300 minutes after the start, respectively. In each image of FIG. 3, the left side is the exposed portion 13 and the right side is the masking portion 12.

As shown in FIG. 3, the color tone of the exposed portion 13 was initially the same as the color tone of the masking portion 12, but changed to a color tone different from that of the masking portion 12 with the elapse of time.

When the constant current polarization is performed, hydrogen is generated in the exposed portion 13 in contact with the electrolytic solution 14, and the hydrogen intrudes the metal material 1. The result of FIG. 3 means that the intrusion of hydrogen into the metal material 1 was captured as the color tone change of the film sensor 2.

Further, after 120 minutes, it could be observed that the color tone of a portion of the exposed portion 13 did not change so much, while a portion where the color tone changed spread beyond a region of the exposed portion 13 to a portion of the masking portion 12.

This means that, with the elapse of time, it was possible to record a state in which hydrogen that had intruded the metal material 1 from the vicinity of a boundary between the exposed portion 13 and the masking portion 12 diffused also in a plane direction of the metal material 1 and spread also in the masking portion 12 that was not in contact with the electrolytic solution 14.

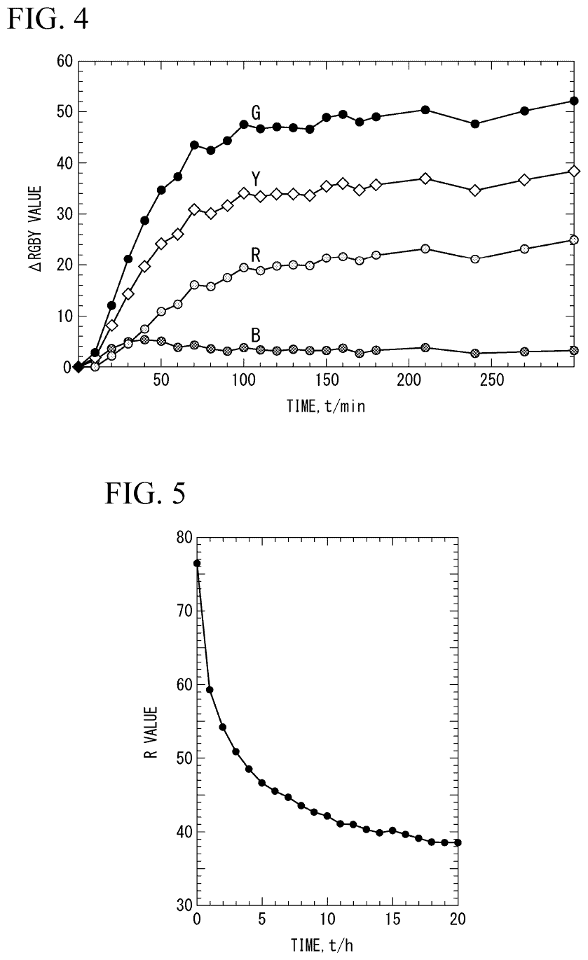

FIG. 4 shows a result of recording the color tone change in the exposed portion 13 from 0 minute to 300 minutes later as an amount of the change of an RGBY value. The RGBY value was respectively an average value of RGBY values of the film sensor 2 in a color tone measurement portion 17 shown in (b) of FIG. 2.

As shown in FIG. 4, the color tone change continued until about 100 minutes later, and thereafter, the color tone change decreased. This indicates that an amount of hydrogen at measurement portion 17 had reached a limit value of an amount of hydrogen detectable with high accuracy by the film sensor 2 about 100 minutes later from the start of measurement.

### Experimental Example 2

After the experiment of Experimental Example 1 was performed, the constant current polarization was further continued, and when the constant current polarization was ended after 600 minutes had elapsed from the start of the constant current polarization, the color tone change at the color tone measurement portion 17 of the exposed portion 13 after the end of the constant current polarization (0 hour) was recorded. The results are shown in FIG. 5.

As shown in FIG. 4, an R value increases while the constant current polarization continues, but as shown in FIG. 5, the R value decreases after the 600 minutes of constant current polarization ends. After the constant current polarization is ended, the amount of hydrogen released from the metal material 1 is larger than the amount of hydrogen intruding the metal material 1, so that the hydrogen concentration in the metal material 1 decreases. The result of FIG. 5 means that the decrease in hydrogen concentration in the metal material 1 was understood as the color tone change of the film sensor 2.

### Experimental Example 3

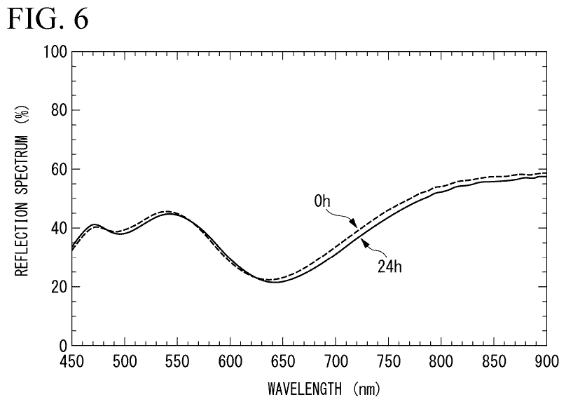

In the apparatus of FIG. 2, the color tone change of the film sensor 2 in a state where the electrolytic solution 14 was not used and the potentiostat 16 was not operated was examined as a reflection spectrum. The result is shown in FIG. 6.

As shown in FIG. 6, the reflection spectrum of the film sensor 2 hardly changed even after 24 hours.

Accordingly, it was confirmed that the color tone change obtained in FIGS. 3 to 5 was not an atmospheric influence on the film sensor 2.

### Experimental Example 4

The constant current polarization was performed for 3 hours in the same manner as in Experimental Example 1, except that the light source of the ring light 5 was changed by using the hydrogen detection system of FIG. 2, and the film sensor 2 of each Example shown below was used instead of the film sensor 2 of Experimental Example 1, so that sensitivity of the film sensor of each Example was evaluated.

As the light source of the ring light 5, a blue LED (emission wavelength 470 nm) was used as the excitation light in Examples 13 to 15, and a xenon lamp was used in the other examples.

The evaluation was performed according to the following criteria based on a respective difference of the RGB values (ΔR, ΔG, ΔB) at the color tone measurement portion 17 between before the start of the constant current polarization and after the constant current polarization was performed for 3 hours. The result is shown in Table 1.

### Evaluation Criteria

5: The maximum value of ΔR, ΔG, and ΔB is 100 or more.
4: The maximum value of ΔR, ΔG, and ΔB is 50 or more and less than 100.
3: The maximum value of ΔR, ΔG, and ΔB is 10 or more and less than 50.
2: The maximum value of ΔR, ΔG, and ΔB is 3 or more and less than 10.
1: The maximum value of ΔR, ΔG, and ΔB is less than 3, or there is no change.

### Example 1

50 mL of an aqueous solution of 0.5 M sulfuric acid was prepared using sulfuric acid (Fuji Film Wako Pure Chemical Corporation, JIS Special Grade), and 1 ml of aniline (Fuji Film Wako Pure Chemical Corporation, JIS Special Grade) and 0.05 g of methyl red (Fuji Film Wako Pure Chemical Corporation, JIS Special Grade) were mixed and stirred until the mixture is completely dissolved to prepare a mixed solution. Here, M represents a molar concentration by volume (mol/L).

The metal material 1 on which the back surface and a portion other than the electrode surface (2 cm square) of the front surface were coated with an acid resistant tape (Electroplating Tape 470, manufactured by 3M Japan Ltd.) was immersed in the mixed solution, and constant-current anode polarization of 3 mA was performed for 3 minutes using the platinum wire as the counter electrode.

A film having a thickness of about 1 μm formed on the electrode surface as the surface of the metal material 1 was sufficiently washed with highly pure water, dried by being blown with nitrogen gas, and then naturally dried at room temperature for 24 hours in the atmospheric environment to obtain the film sensor 2 including of a solid polyaniline layer containing methyl red.

### Example 2

The film sensor 2 including a solid polythiophene layer containing methyl red was prepared in the same manner as in Example 1 except that 1 mL of thiophene (Fuji Film Wako Pure Chemical Corporation, JIS Special Grade) was used instead of 1 mL of aniline.

### Example 3

The film sensor 2 including a solid polypyrrole layer containing methyl red was prepared in the same manner as in Example 1 except that 1 mL of pyrrole (Fuji Film Wako Pure Chemical Corporation, JIS Special Grade) was used instead of 1 mL of aniline.

### Example 4

The film sensor 2 including a solid polyaniline layer containing phenolphthalein was prepared in the same manner as in Example 1 except that 0.05 g of phenolphthalein (Fuji Film Wako Pure Chemical Corporation, JIS Special Grade) was used instead of 0.05 g of methyl red.

### Example 5

The film sensor 2 including a solid polythiophene layer containing phenolphthalein was prepared in the same manner as in Example 1 except that 1 mL of thiophene (Fuji. Film Wako Pure Chemical Corporation, JIS Special Grade) was used instead of 1 mL of aniline and 0.05 g of phenolphthalein (Fuji Film Wako Pure Chemical Corporation, JIS Special Grade) was used instead of 0.05 g of methyl red.

### Example 6

The film sensor 2 including a solid polypyrrole layer containing phenolphthalein was prepared in the same manner as in Example 1 except that 1 mL of pyrrole (Fuji Film Wako Pure Chemical Corporation, JIS Special Grade) was used instead of 1 mL of aniline and 0.05 g of phenolphthalein (Fuji Film Wako Pure Chemical Corporation, JIS Special Grade) was used instead of 0.05 g of methyl red.

### Example 7

The film sensor 2 including a solid polyaniline layer containing p-nitrophenyl hydrazone was prepared in the same manner as in Example 1 except that 0.05 g of p-nitrophenyl hydrazone (manufactured by Fluorochem Ltd., 4-nitrophenylhydrazine hydrochloride) was used instead of 0.05 g of methyl red.

### Example 8

The film sensor 2 including a solid polythiophene layer containing p-nitrophenyl hydrazone was prepared in the same manner as in Example 1 except that 1 mL of thiophene (Fuji Film Wako Pure Chemical Corporation, JIS Special Grade) was used instead of 1 mL of aniline and 0.05 g of p-nitrophenyl hydrazone (manufactured by Fluorochem Ltd., 4-nitrophenylhydrazine hydrochloride) was used instead of 0.05 g of methyl red.

### Example 9

The film sensor 2 including a solid polypyrrole layer containing p-nitrophenyl hydrazone was prepared in the same manner as in Example 1 except that 1 mL of pyrrole (Fuji Film Wako Pure Chemical Corporation, JIS Special Grade) was used instead of 1 mL of aniline and 0.05 g of p-nitrophenyl hydrazone (manufactured by Fluorochem Ltd., 4-nitrophenylhydrazine hydrochloride) was used instead of 0.05 g of methyl red.

### Example 10

The film sensor 2 including a solid polyaniline layer containing alizarin yellow was obtained in the same manner as in Example 1 except that 0.05 g of alizarin yellow (Fuji Film Wako Pure Chemical Corporation, alizarin yellow R, JIS Special Grade) was used instead of 0.05 g of methyl red.

### Example 11

The film sensor 2 including a solid polythiophene layer containing alizarin yellow was prepared in the same manner as in Example 1 except that 1 mL of thiophene (Fuji Film Wako Pure Chemical Corporation, JIS Special Grade) was used instead of 1 mL of aniline, and 0.05 g of alizarin yellow (Fuji Film Wako Pure Chemical Corporation, alizarin yellow R, JIS Special Grade) was used instead of 0.05 g of methyl red.

### Example 12

The film sensor 2 including a solid polypyrrole layer containing alizarin yellow was prepared in the same manner as in Example 1 except that 1 mL of pyrrole (Fuji Film Wako Pure Chemical Corporation, JIS Special Grade) was used instead of 1 mL of aniline, and 0.05 g of alizarin yellow (Fuji Film Wako Pure Chemical Corporation, alizarin yellow R, JIS Special Grade) was used instead of 0.05 g of methyl red.

### Example 13

The film sensor 2 including a solid polyaniline layer containing sodium fluorescein was prepared in the same manner as in Example 1 except that 0.05 g of sodium fluorescein (Fuji Film Wako Pure Chemical Corporation, Uranine, JIS Special. Grade) was used instead of 0.05 g of methyl red.

### Example 14

The film sensor 2 including a solid polythiophene layer containing sodium fluorescein was prepared in the same manner as in Example 1 except that 1 mL of thiophene (Fuji Film Wako Pure Chemical Corporation, JIS Special Grade) was used instead of 1 mL of aniline and 0.05 g of sodium fluorescein (Fuji Film Wako Pure Chemical Corporation, Uranine, JIS Special Grade) was used instead of 0.05 g of methyl red.

### Example 15

The film sensor 2 including a solid polypyrrole layer containing sodium fluorescein was prepared in the same manner as in Example 1 except that 1 mL of pyrrole (Fuji Film Wako Pure Chemical Corporation, JIS Special Grade) was used instead of 1 mL of aniline and 0.05 g of sodium fluorescein (Fuji Film Wako Pure Chemical Corporation, Uranine, JIS Special Grade) was used instead of 0.05 g of methyl red.

### Example 16

3 g of agar (Fuji Film Wako Pure Chemical Corporation, JIS Special Grade) was dissolved in 50 ml of a saturated solution of KCl (Fuji Film Wako Pure Chemical Corporation, JIS Special Grade) heated to 90°° C. to obtain an agar solution, and 50 ml of the obtained agar solution was then mixed with 0.05 g of methyl red (Fuji Film Wako Pure Chemical Corporation, JIS Special Grade), and stirred until completely dissolved to prepare a mixed solution.

The mixed solution of agar and methyl red was added dropwise to the metal material 1 on which the entire back surface and a portion other than the electrode surface (2 cm square) of the front surface were coated with an acid resistant tape (Electroplating Tape 470, manufactured by 3M Japan Ltd.), and the mixture was dried at room temperature so as to form a film, thereby obtaining the film sensor 2 including a solid agar layer containing methyl red.

### Example 17

The film sensor 2 including a solid agar layer containing phenolphthalein was prepared in the same manner as in Example 16 except that 0.05 g of phenolphthalein (Fuji Film Wako Pure Chemical Corporation, JIS Special Grade) was used instead of 0.05 g of methyl red.

### Example 18

The film sensor 2 including a solid polythiophene layer containing no hydrogen functional dye was obtained in the same manner as in Example 2 except that methyl red was not added.

### Example 19

The film sensor 2 including a solid polypyrrole layer containing no hydrogen functional dye was obtained in the same manner as in Example 3 except that methyl red was not added.

TABLE 1

| | | | Sensitivity |
|---|---|---|---|
| Number | Hydrogen functional dye | Organic polymer | evaluation |
| Example 1 | Methyl red | Polyaniline | 5 |
| Example 2 | Methyl red | Polythiophene | 4 |
| Example 3 | Methyl red | Polypyrrole | 5 |
| Example 4 | Phenolphthalein | Polyaniline | 5 |
| Example 5 | Phenolphthalein | Polythiophene | 4 |
| Example 6 | Phenolphthalein | Polypyrrole | 3 |
| Example 7 | p-nitrophenylhydrazone | Polyaniline | 5 |
| Example 8 | p-nitrophenylhydrazone | Polythiophene | 4 |
| Example 9 | p-nitrophenylhydrazone | Polypyrrole | 3 |
| Example 10 | Alizarin yellow | Polyaniline | 5 |
| Example 11 | Alizarin yellow | Polythiophene | 5 |
| Example 12 | Alizarin yellow | Polypyrrole | 4 |
| Example 13 | Sodium fluorescein | Polyaniline | 5 |
| Example 14 | Sodium fluorescein | Polythiophene | 4 |
| Example 15 | Sodium fluorescein | Polypyrrole | 3 |
| Example 16 | Methyl red | Agar | 2 |
| Example 17 | Phenolphthalein | Agar | 2 |
| Example 18 | — | Polythiophene | 3 |
| Example 19 | — | Polypyrrole | 2 |

As shown in Table 1, it could be confirmed that hydrogen can be detected by using various hydrogen functional polymers and hydrogen functional dyes.

### Experimental Example 5

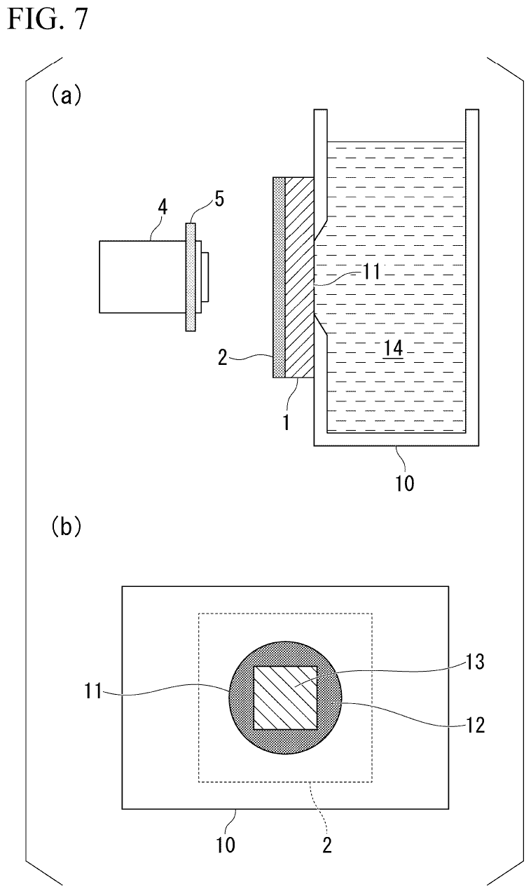

It was confirmed that corrosion of a metal material exposed to an electrolytic solution can be observed by a hydrogen detection system of FIG. 7.

The hydrogen detection system of FIG. 7 includes the film sensor 2 laminated on one surface of the metal material 1, the camera 4 for imaging the film sensor 2 from the side (front side) opposite from the metal material 1, and the ring light 5 attached to the camera 4. A single-lens reflex camera was used as the camera 4. A halogen lamp was used as a light source of the ring light 5.

Then, the surface (back surface) of the metal material 1 opposite from the surface on which the film sensor 2 was laminated was pressed to the position where the cell opening 11 was to be closed.

Further, the liquid tightness is maintained between the back surface of the metal material 1 and the acrylic cell 10 by the sealing member (not shown) surrounding the cell opening 11.

The acrylic cell 10 was filled with the 3 mass % NaCl aqueous solution as the electrolytic solution 14.

In the present experimental example, as shown in (b) of FIG. 7, the center of the portion of the metal material 1 facing the cell opening 11 was set as the rectangular exposed portion 13 (width 10 mm×length 10 mm), and an outside thereof was set as the masking portion 12 (diameter 16 mm).

The film sensor 2 was formed on the metal material 1 in the same manner as in Experimental Example 1.

As the metal material 1, a material same as the metal material 1 of Experimental Example 1 was used, and the masking portion 12 was formed in the same manner as in Experimental Example 1.

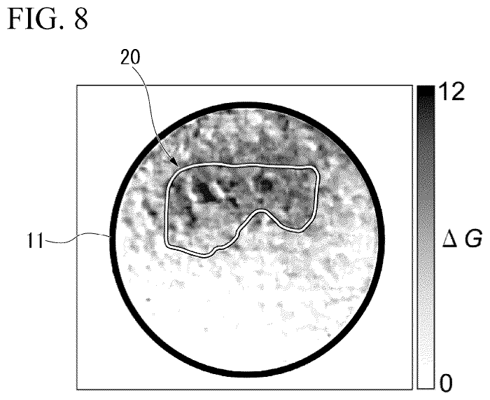

FIG. 8 shows an image captured by the camera 4 in 24 hours after the exposed portion 13 of the metal material 1 is exposed to the electrolytic solution 14. In the image of FIG. 8, the change in the G value (ΔG) is shown by shading of black and white.

As shown in FIG. 8, a discolored portion 20 was observed in a portion corresponding to substantially an upper half of the exposed portion 13. When the exposed portion 13 on the back surface of the metal material 1 was observed, corrosion was observed in a portion corresponding to the discolored portion 20.

When the corrosion of the metal material 1 occurs at an interface with the electrolytic solution, hydrogen is generated. The result of the present experimental example means that the increase in the hydrogen concentration due to the corrosion of the metal material 1 was understood as the color tone change of the film sensor 2.

### INDUSTRIAL APPLICABILITY

The film sensor and the hydrogen detection system according to the invention can be applied to the detection of hydrogen present in a metal material used in pipes for a liquid or a gas, containers such as a high-pressure hydrogen gas cylinder and a natural gas cylinder, and transport machines such as automatic vehicles.

### REFERENCE SIGNS LIST

1 metal material
2 film sensor
4 camera
5 ring light
10 acrylic cell
11 cell opening
12 masking portion
13 exposed portion
14 electrolytic solution
15 counter electrode
16 potentiostat
20 discolored portion

1. A film sensor for detecting hydrogen in a metal material by being laminated on a surface of the metal material in a gas or in vacuum, wherein
the film sensor is formed of an organic polymer whose optical characteristic changes depending on a hydrogen concentration.

2. The film sensor according to claim 1, wherein
the organic polymer whose optical characteristic changes depending on the hydrogen concentration is a conjugated polymer.

3. The film sensor according to claim 2, wherein
the organic polymer whose optical characteristic changes depending on the hydrogen concentration is an aromatic conjugated polymer.

4. The film sensor according to any one of claims 1 to 3, wherein
the organic polymer whose optical characteristic changes depending on the hydrogen concentration has at least one of an amine structure or an imine structure.

5. The film sensor according to claims 1 to 4, wherein
the film sensor contains a dye whose optical characteristic changes depending on the hydrogen concentration.

6. A film sensor for detecting hydrogen in a metal material by being laminated on a surface of the metal material in a gas or in vacuum, wherein
the film sensor contains a dye which is formed of an organic polymer and whose optical characteristic changes depending on a hydrogen concentration.

7. A hydrogen detection system comprising:
the film sensor according to any one of claims 1 to 6; and
an optical detector configured to detect an optical characteristic of the film sensor.
